(19) United States
(12) Patent Application Publication
(10) Pub. No.: US 2004/0180865 A1
(43) Pub. Date: Sep. 16, 2004

(54) ASPIRIN AND VITAMIN AND/OR TRACE ELEMENT COMPOSITIONS FOR THE AMELIORATION AND TREATMENT OF VASCULAR DISEASE

(76) Inventor: JEFFRY W. KREAMER, BUFFALO GROVE, IL (US)

Correspondence Address: DAVIS, BROWN, KOEHN, SHORS & ROBERTS, P.C., THE FINANCIAL CENTER, 666 WALNUT STREET, SUITE 2500, DES MOINES, IA, 50309-3993, US

(21) Appl. No.: 08/071,052

(22) Filed: Jun. 4, 1993

(51) Int. Cl. A61K 31/60

(52) U.S. Cl. 514/165

(57) ABSTRACT

A medicament for the amelioration and treatment of vascular disease consisting of aspirin administered in combination with one or more of the vitamins including vitamin A, vitamin B₆, vitamin C, vitamin E, and niacin. Alternative embodiments include the administration of aspirin in combination with one or more trace element including selenium, zinc, copper, iron, cobalt, and manganese as well as combinations of aspirin, at least one vitamin and at least one trace element.

### BACKGROUND OF THE INVENTION

The invention relates generally to a composition useful in treating vascular disease and, more specifically, to aspirin combined with one or more vitamins and in combination or separately with one or more trace elements and used in the amelioration and treatment of vascular disease.

The effects of aspirin in the amelioration and treatment of certain types of vascular disease (e.g., atherosclerosis and other lumen decreasing diseases) have recently been documented. Although the full range of effects on the human body of low-level dosages of aspirin taken over an extended period are still being investigated, low-level dosages of aspirin are being prescribed by many physicians to their male patients with high-risk indications for vascular disease and who do not have a history of stroke or aneurysm. Aspirin has been recognized since the late 60's as a treatment for atherosclerotic disease. Harker, L. A., Circulation 73 (2): 206-223 (1986).

The administration of vitamin combinations in the amelioration and treatment of atherosclerosis has been the subject of research beginning in about the early 50's. Simonon, E., et al. Circulation 24: 1239-1248 (1961).

More recently ameliorative effects on atherosclerosis has been observed in certain trace elements or minerals, such as selenium, magnesium, chromium, zinc, and copper. Virtamo, J. and Huttunen, J. K., Ann. Clin. Res. 20:102-113 (1988) The effects of individual vitamins on the circulatory system has also been the subject of considerable study. Niacin (nictotinic acid) has been shown to reduce the blood serum levels of cholesterol and triglycerides resulting in a decreased incidence of the end events of atherosclerosis. Conner, P. J., et al. (1986) Fifteen-Year in Coronary Drug Project Patients: Long-term Benefit with Niacin. J.A.C.C. 8: 1245-125. Vitamin C increases the level of activity of prostaglandins in the vascular wall. Beetens, J. R., et al. (1984) Influence of Vitamin C on the Metabolism of Arachatonic Acid and the Development of Aortic Lesions During Experimental Atherosclerosis in Rabbits. Biomed. Biochim. Acta 43: 5273-5276. Vitamin E is thought to exert a protective effect on immune-triggered endothelial damage. Boogaerts, M. A., et al. (1984) Protective Effect of Vitamin E on Immune-triggered, Granulocyte-medicated Endothelial Injury. Thromb. Haemost. 51: 89-92. Vitamin A speeds vascular wall wound healing probably through enhanced collagen accumulation. Niu, X. T., et al. (1987) Effect of Dietary Supplementation with Vitamin A on Arterial Healing in Rats. J. Surg. Res. 42: 61-65. A deficiency in vitamin B₆ leads to formation of atherosclerotic plaques due to inefficient cholesterol handling. Koumans, A. K., et al. (1985) Nutrition and Atherosclerosis: Some Neglected Aspects. Clin. Cardiol. 8: 547-551.

Rats that have been depleted in serum selenium have been reported to have increased platelet aggregation and decreased prostacyclin production. Bult, H., et al., Thromb. Haemost. 46:272 (1981); Masukawa, T., et al., Experientia 39:405-6 (1983); Schoene, N. W., et al., Nutr. Res. 6:75-83 (1986). A significantly reduced serum selenium level in subjects with coronary heart disease was demonstrated in two studies. Oster, O., et al., Ann. Clin. Res. 18:36-42 (1986); Moore, J. A., et al., Clin. Chem. 30:1171-3 (1984). Magnesium deficiency has been reported to have several effects, including initial thickening, calcification, and collagen accumulation in the vascular wall, an increase in very low and low density lipoprotein cholesterols and a decrease in high density lipoprotein cholesterol, and increases the susceptibility of platelets to thrombin-induced aggregation. Rayssiguier, Y. and Gueux, E., J. Am. Coll. Nutr. 5:507-19 (1986). Elevated serum cholesterol levels and the deposition of aortic placques in rats were prevented by the ameriolation of chromium deficiency. Schroeder, H. A., Am. J. Clin. Nutr. 21:230-44 (1968). Studies on middle-aged men from Finland suggested that coronary heart disease may be associated with a low concentration of chromium in the drinking water. Punsar, S., et al., J. Chron. Dis. 28:259-87 (1075); Punsar, S. and Karvoner, M. J., Cardioloqy 64:24-34 (1979). With respect to the zinc, hyperlipoproteinemic subjects with symptoms of atherosclerosis were found to have lower serum zinc levels than did asympomatic hyperlipoproteinemic subjects, Uza, G., Biol. Trace Element Res. 8:167-72 (1985), and a diet containing seven times the level of zinc required for normal growth greatly reduced in rats the incidence and severity of vascular lesions in branches of the aorta and cerebral arteries. Petering, H. G., et al., Biol. Trace Element Res. 9:251-70 (1986). Copper deficiency has been suggested as being involved in the impaired function of lysyl oxide and leading to atherosclerosis. Allen, K. G. D. and Klevay, L. M., Atherosclerosis 29:81-93 (1978). Copper deficiency increases plasma lipids in rats and the hypercholesterolemic effect of copper deficiency has been attributed to rapid synthesis and clearance of cholesterol into the plasma pool. Ibid.; and Klevay, L. M., Nutr. Rep. Int. 22:259-9 (1980).

In accordance with the present invention, aspirin is combined with one or more vitamins. The activity of the vitamins on vascular wound healing and the formation of atherosclerotic plaque combine synergistically with the beneficial effects of aspirin. Moreover, the same or other vitamins have activities which are ameliorative or antagonistic to some of the deleterious effects of aspirin. Combinations of aspirin and vitamins increase the prophylactic and therapeutic effects on vascular disease more than either aspirin or the vitamins individually. Combinations of aspirin and one or more trace elements, alone or together with one or more vitamins, is also taught.

### SUMMARY OF THE INVENTION

Compositions of the present invention are a pharmaceutical or medicament made of aspirin and at least one vitamin selected from the group consisting of vitamin A, vitamin B₆, vitamin C, vitamin E, and niacin, which compositions are administered for the prevention or treatment of vascular disease wherein the vitamin component(s) is present in up to about the United States Recommended Daily Allowance (R.D.A.). In an alternative embodiment, the medicament is comprised of aspirin and at least one trace element selected from the group including selenium, zinc, copper, iron, cobalt and manganese wherein the mineral component is present in amounts up to about the U.S.R.D.A. The alternative medicament may also include one or more vitamins. In a preferred regimen, the composition is administered orally once a day.

### DETAILED DESCRIPTION OF A PREFERRED EMBODIMENT

The mechanisms of vascular wound healing and formation of atherosclerotic plaques are affected by aspirin and a number of vitamins. It is currently believed that in the process of ordinary living the lining or endothelium of blood vessels is undergoing constant injury and healing. The injuries, often microscopic, are caused by a number of processes, including the normal pressure of blood inside the vessel, peroxidation, and immune complexes. As these wounds heal, cholesterol invades the media or middle layer of the vascular wall. Plaque formed of the cholesterol causes the media to bulge and thus encroach upon the arterial lumen. Myocardial infarction can result due to advanced plaque formation in the cardiac arterial system causing decreased blood flow and therefore decreased oxygen delivery.

The normal, main healing process of the microscopic wounds starts with increased platelet activity. The platelets migrate to the site of the endothelial lesion and start thrombosis or clot formation. As the healing process progresses, blood, of course, continues to flow through the vessel and over the site of the lesion. Cholesterol and triglycerides carried in the blood (serum cholesterol) become incorporated in the clotting matrix and then migrate through the intima to the media. Incorporation of cholesterol in the endothelium is thought to be the principal start of atherosclerotic plaque formation. The lower the level of serum cholesterol and triglycerides, or the faster secondary wound healing occurs, the less cholesterol will be able to accumulate in the vascular wall. The normal healing of vascular lesion is promoted by prostaglandin function in the vascular wall, lowered levels of serum cholesterol and triglycerides, and an increase in the platelet anti-aggregant effect. Further, the incidence of vascular lesions can be reduced by increasing endogenous anti-oxidant potential and lowering immune-triggered endothelial damage.

Aspirin is an anti-inflammatory agent which is known in the art to irreversibly block platelet protaglandin function. This inhibits the ability of platelets to go to the site of endothelial damage and hence start wound healing by the activity of the platelets themselves and their ability to recruit other cells to assist in thrombosis. Thus, aspirin will inhibit the start of the healing process. But because of the connection between platelet activity and cholesterol migration into the clot, it has the beneficial effect of decreasing the undesirable plaque formation.

Aspirin also reversibly decreases prostaglandin activity in the vascular wall. Prostaglandins in the vascular wall acts to expel cholesterol, thus inhibiting plaque formation. The extent of this effect, however, is less than the reduction in cholesterol migration due to the irreversible blockage of platelet prostaglandin function described above. Aspirin also has a generalized reduction in the prostaglandin effect and reduces the tendency of blood clot formation (through its platelet activity), a principal cause of myocardial infarction. This effect is reported in Young, F. E., et al. (1988) The Preliminary Report of the Findings of the Aspirin Component of the Ongoing Physician's Health Study. J.A.M.A. 259:3158-3160.

Vitamin A has been observed to improve arterial healing by increasing the rate at which reparative collagen accumulates at the lesion in the media. Therefore, vitamin A antagonizes the wound healing inhibitory effect of aspirin. Niu et al., J. Surg. Res. 42, 61-65 (1987). Thus, while aspirin delays cholesterol migration into the clot, vitamin A promotes wound healing. At higher doses, vitamin A may result in a net increase in serum cholesterol levels. Melnik, B. C., et al. (1987) Evaluation of the Atherogenic Risk of Isoretinoin-induced and Etretinate-induced Alterations of Lipoprotien Cholesterol Metabolism. J. Ivest. Dermatol. 88, 395-435. Vitamin A also has a mild anti-aggregant activity which, since it decreases standard clot formation, will decrease the likelihood of cholesterol/clot matrix damage to the arterial wall. Butturini, V., Acta Vitaminol. Enzymol. 4:15-19 (1982).

A deficiency in vitamin B₆ is known to cause inefficiencies in cholesterol handling and thus promote the formation of atherosclerotic plaques. Koumans et al., Clin. Cardiol. 8:547-551 (1984). Vitamin B₆ and aspirin act together to enhance cholesterol handling while decreasing the propensity of cholesterol to invade the lesion healing site.

Prostaglandin levels in the vascular wall are increased by vitamin C. Beetens et al., Biomed. Biochim. Acta 43:5273-5276 (1984). This effect is antagonistic to the effect of aspirin in reversibly lowering prostaglandin activity in the vascular wall. Vitamin C thus counteracts that negative aspect of aspirin to result in enhanced wound healing when administered in combination with aspirin. Increasing prostaglandin activity in the vascular wall will also cooperate with the enhanced cholesterol handling benefit of vitamin B₆ supplementation to promote more efficient wound healing. Another benefit of vitamin C supplementation is seen in the prevention of the mild vitamin C deficiency exhibited in many smokers. Mild vitamin C deficiency causes a decrease in the cholesterol handling ability of the liver with the concomitant increase of serum cholesterol as well as the level of cholesterol in the liver and arteries. Ginter, E., et al., Vitamin C in the Control of Hypocholesterolemia in Man. Int. J. Vitam. Nutr. Res. 23:137-152 (1982).

Of course, a reduction in the incidence of lesions will act to slow atherosclerosis. One source of vascular lesions is immune-induced endothelial damage. Vitamin E and vitamin A have anti-oxidant activity that exert a preventative effect on immune-induced lesions partly through increasing endogenous anti-oxidant potential and partly by modulating the intrinsic endothelial prostaglandin production. Boogaerts et al., Thromb. Haemost. 51:89-92 (1984).

Vitamin E has been shown to reduce the severity of lesions in animals fed an atherogenic diet. Donaldson, W. E., Atherosclerosis in Cholesterol-fed Japanese Quail: Evidence for Amelioration by Dietary Vitamin E. Poult. Sci. 61:2097-2102 (1982). Similar to vitamin A, vitamin E has a mild anti-aggregant effect. Butturini Acta Vitaminol.Enzymol. 4:15-19 (1982). The protective effect of vitamin E on immune-induced lesions may produce an enhanced prophylactic and therapeutic effect when used in combination with vitamin C and its effect on increasing the prostaglandin activity in the vascular wall.

Finally, supplementation of vitamin E in deficient animals has been shown to decrease serum and liver cholesterol levels in a dose- and time-dependent manner. Kaseki, H. et al., Effect of an Oral Dose of Vitamin E on the Vitamin E and Cholesterol Content of Tissues of the Vitamin E-Deficient Rate. J. Nutr. 116:1631-1639 (1986).

Supplements of niacin (nictotinic acid) in sufficient amounts are known to cause skin flush and dizziness. The skin flush is mediated by prostaglandins in the blood vessel walls. Wilson, D. W. S. and Douglass, A. B., Niacin Skin Flush is Not Diagnostic of Schizophrenia. Biol. Psychiatry 21:974-977 (1986). The skin flush and dizziness caused by niacin are uncomfortable, though harmless; aspirin blocks these specific prostaglandins and will lessen the undesirable effects of niacin. Serum cholesterol and triglyceride levels are reduced by niacin. Canner, et al., J.A.C.C. 8:1245-1255 (1986). Thus niacin can be used alone as a potent cholesterol-lessening agent or, when used in combination with vitamin A, will mitigate its effect on increasing cholesterol levels, and will assist in reducing cholesterol migration into the clotting matrix of healing vascular lesions by decreasing the available cholesterol, especially when used in combination with aspirin.

Selenium works as an anti-oxidant. A low level has been shown to increase low density lipoprotein cholesterols. Stone, W. L., Ann. Nutr. Metab. 30:94-103 (1986) and increase platelet reactivity. Bult, H., Thromb. Heamost 46:272 (1981); Masukawa, T., Experientia 39:405-6 (1983); Schoene, N. W., Nutr. Res. 6:75-83 (1986). It has been shown that very low levels are an independent risk factor for atherosclerosis. Oster, O, Ann. Clin. Res. 18:36-42 (1986); Wang, Y. X., Klin. Wochenschr 59:187-8 (1981); Salonen, J. T., Lancet ii: 175-9 (1992). Supplementation of selenium will have a beneficial interaction with other anti-oxidants (such as Vitamin E), cholesterol modifiers (Vitamin B₆, niacin), and platelet activity decreasers (aspirin) and will participate with the previously mentioned group interactions.

Magnesium exerts its effects of atherosclerosis by many means. Deficient states can cause intimal thickening, thinning and fragmentation of elastic membranes, collagen accumulation, increase in LDL cholesterol, a decrease in HDL cholesterol, and can make platelet activity hyperactive to the vascular wall. Rayssiguier, Y. and Gueux, E., J. Am. Coll. Nutur. 5:507-19 (1986) Magnesium will thus have a positive interaction with cholesterol modifiers, wall healers (Vitamin A) platelet activity decreasers, as well as all previously mentioned inter-group interactions.

Chromium deficiency leads to increased cholesterol Schroeder, H. A., Am. J. Clin. Nutr. 21:230-44 (1968); Vinson, J. A. and Bose, P., Nutr. Rep. Int. 30:911-8 (1984); Elwood, J. C., J. Am. Coll. Nutr. 1:263-74 (1982); Riales, R. Am. J. Clin. Nutr. 34:2670-8 (1981) and Offenbacher, E. G. and Pi-Sunyer, F. X., Diabetes 29:919-25 (1980) and it has been found that serum chromium was lower in people with coronary heart disease. Newman, H. A. I., Clin. Chem. 24:541-4 (1978) Chromium supplementation thus will work in harmony with cholesterol modifiers and their previously mentioned interactions.

It is unclear how zinc exerts influence in atherosclerosis. However, it has been shown that acute zinc depletion causes decreased HDL cholesterol Koo, S. I., Am. J. Clin. Nutr. 34:2376-81 (1981) and a higher zinc diet shows reduced incidence of atherosclerosis in rats. Petering, H. G., Biol. Trace Elements Res. 9:251-70 (1986) As with chromium, zinc supplementation will work in harmony with cholesterol modifiers and their previously mentioned interactions.

Copper exerts its influence on atherosclerosis by mediating the cross linking and structural integrity of vascular connective tissue. Allen, K. G. D. and Klevay, L. M., Atherosclerosis, 29:81-93 (1978) Deficiency also causes increased cholesterol Allen, K. G. D. and Klevay, L. M., Atherosclerosis, 29:81-92 (1978); Allen, K. G. D. and Klevay, L. M., Atherosclerosis 31:259-71 (1978) and Allen, K. G. D. and Klevay, L. M., Nutr. Rep. Int. 22:295-9 (1980) probably due to ineffective handling of the cholesterol. Copper supplementation will thus work in harmony with wall healers and cholesterol modifiers and their previously mentioned interactions.

Tables 1 and 2 summarize the effects of aspirin and the six considered vitamins and five considered trace elements on nine aspects of vascular lesions and healing that impact the health of vascular tissue. It can be seen that the deleterious effects of aspirin can be largely overcome or ameliorated by the administration of one or more of the six vitamins and five trace elements.

The efficacy of the vitamins for the prevention and treatment of atherosclerosis and other non-structural forms of vascular disease has been observed at dosages up to about the United States Recommended Daily Allowance (R.D.A.).

The medicaments of the present invention include combinations of aspirin and at least one vitamin selected from the group including vitamin A, vitamin B₆, vitamin C, vitamin E, and niacin wherein the aspirin and vitamin or vitamins are present in amounts effective for the amelioration and treatment of vascular disease. The amount of aspirin is between about 20 mg and about 325 mg and the amount of each vitamin is between about 10% and about 200% of the U.S.R.D.A. for each vitamin, considered on an average daily dose basis. In the preferred embodiment, between about 40 mg. and about 100 mg. of aspirin combined with between about 50% and about 100% of the U.S.R.D.A. of one or more of the vitamins is administered orally on a daily basis. More or less frequent administration of the medicaments can be made with a corresponding adjustment in the dosage.

In an alternative embodiment, the medicaments include combinations of aspirin and at least one trace element selected from the group including selenium, zinc, iron, copper, cobalt, and manganese wherein the aspirin and trace element or elements are present in amounts effective for the amelioration and treatment of vascular disease. The amount of aspirin is between about 20 mg and about 325 mg and the amount of each trace element is between about 10% and about 200% of the U.S.R.D.A. for each trace element, considered on an average daily dose basis. In the preferred embodiment, between about 40 mg. and about 100 mg. of aspirin combined with between about 50% and about 100% of the U.S.R.D.A. of one or more of the trace elements is administered orally on a daily basis. More or less frequent administration of the medicaments can be made with a corresponding adjustment in the dosage.

In a second alternative embodiment, the medicaments include combinations of aspirin and at least one of the listed vitamins, and at least one of the listed trace elements, in amounts and dosages as discussed above.

Table 3 summarizes the results of an unpublished observational study conducted by the University of Southern California of 13,987 retirement community residents, 8881 women and 5106 men with a median age of 73. In men, aspirin plus a multivitamin showed a marked relative risk (RR) reduction in all deaths, total cardiovascular disease, acute myocardial infarction, ischemic heart disease, and stroke. In women, deaths were below baseline in all categories and was the lowest in all deaths, total cardiovascular disease, other heart disease, and stroke. This study demonstrates an unexpected synergy effect of an aspirin and multivitamin medicament on atherosclerosis which is greater than any single component or simple additive effects from more than one component. Relative risk reductions were greatest at an administration rate of one aspirin weekly.

Although the invention has been described with respect to a preferred embodiment thereof, it is to be also understood that it is not to be so limited since changes and modifications can be made therein which are within the full intended scope of this invention as defined by the appended claims.

TABLE 1

| | Aspirin | Vitamin A | Vitamin B₆ | Vitamin C | Vitamin E | Niacin |
|---|---|---|---|---|---|---|
| Platelet Activity | − | ∘ | ∘ | ∘ | ∘ | ∘ |
| Prostaglandin | − | ∘ | ∘ | + | + | + |
| Synthesis | | | | | | |
| Decreases Serum | ∘ | − | + | + | + | + |
| Cholesterol | | | | | | |
| Inhibition of | + | ∘ | ∘ | ∘ | ∘ | ∘ |
| Cholesterol Migration | | | | | | |
| Decreased Formation | + | ∘ | ∘ | ∘ | ∘ | ∘ |
| of Clots | | | | | | |
| Speeding of Wound | − | + | ∘ | ∘ | ∘ | ∘ |
| Healing | | | | | | |
| Anti-aggregant | + | + | ∘ | ∘ | + | ∘ |
| Decreased Immune- | ∘ | ∘ | ∘ | ∘ | + | ∘ |
| Induced Lesions | | | | | | |
| Decreased | ∘ | ∘ | ∘ | ∘ | + | ∘ |
| Peroxidation | | | | | | |

+ beneficial
∘ no effect
− detrimental

TABLE 2

| | Chromium | Copper | Magnesium | Selenium | Zinc |
|---|---|---|---|---|---|
| Platelet Activity | ∘ | ∘ | + | + | ∘ |
| Prostaglandin | ∘ | ∘ | ∘ | + | ∘ |
| Synthesis | | | | | |
| Decreases Serum | + | + | + | + | + |
| Cholesterol | | | | | |
| Inhibition of | ∘ | ∘ | ∘ | ∘ | ∘ |
| Cholesterol | | | | | |
| Migration | | | | | |
| Decreased | ∘ | ∘ | ∘ | ∘ | ∘ |
| Formation | | | | | |
| of Clots | | | | | |
| Speeding of | ∘ | + | + | ∘ | ∘ |
| Wound | | | | | |
| Healing | | | | | |
| Anti-aggregant | ∘ | ∘ | ∘ | + | ∘ |
| Decreased | ∘ | + | ∘ | + | ∘ |
| Immune- | | | | | |
| Induced | | | | | |
| Lesions | | | | | |
| Decreased | ∘ | ∘ | ∘ | + | ∘ |
| Peroxidation | | | | | |

+ beneficial
∘ no effect
− detrimental

1. (Canceled)

2. (Canceled)

3. (Canceled)

4. (Canceled)

5. (Canceled)

6. (Canceled)

7. (Canceled)

8. (Canceled)

9. (Canceled)

10. (Canceled)

11. (Canceled)

12. (Canceled)

13. (Canceled)

14. (Canceled)

15. (Canceled)

16. (Canceled)

17. (Canceled)

18. (Canceled)

19. (Canceled)

20. (Canceled)

21. (Canceled)

22. (Canceled)

23. (Canceled)

24. (Canceled)

25. (Canceled)

26. (Canceled)

27. A medicament for reducing atherosclerotic plaque formation at sites of endothelial damage in humans through the reduction of cholesterol incorporation into the endothelium comprising:
(a) aspirin orally administered in a dose effective to block prostaglandin function in platelets sufficiently to reduce the ability of platelets to go to the site of the endothelial damage and recruit other cells to assist in thrombosis; and
(b) a vitamin orally administered in a dose effective to reduce the migration of cholesterol into the endothelium, wherein said multivitamin comprises at least one vitamin from the group including niacin, vitamin C, vitamin E, and vitamin B₆.

28. A medicament as defined in claim 27, wherein said multivitamin contains between about 10 percent and 200 percent of the United States Recommended Daily Allowance of at least one vitamin selected from the group consisting of vitamin B₆, vitamin C, vitamin E, and niacin.

29. A medicament as defined in claim 27, wherein said effective dose of aspirin is between about 20 milligrams and about 325 milligrams of aspirin per day.

30. A medicament for reducing atherosclerotic plaque formation at sites of endothelial damage in humans through the reduction of cholesterol incorporation into the endothelium comprising:
(a) aspirin orally administered in a dose effective to block prostaglandin function in platelets sufficiently to reduce the ability of platelets to go to the site of the endothelial damage and recruit other cells to assist in thrombosis; and
(b) a vitamin orally administered in a dose effective to reduce the migration of cholesterol into the endothelium, wherein said multivitamin comprises at least one vitamin from the group including niacin, vitamin C, vitamin E, and vitamin B₆.

31. A medicament as defined in claim 30, wherein said multivitamin contains between about 10 percent and 200 percent of the United States Recommended Daily Allowance of at least one vitamin selected from the group consisting of vitamin B₆, vitamin C, vitamin E, and niacin.

32. A medicament as defined in claim 30, wherein said effective dose of aspirin is between about 20 milligrams and about 325 milligrams of aspirin per day.
